(19) United States
(12) Patent Application Publication
(10) Pub. No.: US 2024/0156626 A1
(43) Pub. Date: May 16, 2024

(54) STENT WITH REMOTE MANIPULATION

(71) Applicant: Merit Medical Systems, Inc., South Jordan, UT (US)

(72) Inventors: Mattias Nielsen, North Salt Lake, UT (US); John Hall, Bountiful, UT (US); Christopher Cindrich, Highland, UT (US)

(21) Appl. No.: 18/507,711

(22) Filed: Nov. 13, 2023

Related U.S. Application Data
(60) Provisional application No. 63/383,621, filed on Nov. 14, 2022.

(51) Int. Cl. A61F 2/954; A61F 2/07

(52) U.S. Cl. CPC A61F 2/954; A61F 2/07; A61F 2002/061

(57) ABSTRACT

A vascular stent for treating a patient that can be manipulating remotely when the vascular stent is in a compressed configuration or in an expanded configuration. The vascular stent comprises a plurality of strings that can be used to open fenestrations on the vascular stent, manipulate the frame of the vascular stent, or shift the vascular stent within a vessel of a patient.

### RELATED APPLICATIONS

This application claims priority to U.S. Provisional Application No. 63/383,621, filed on Nov. 14, 2022 and titled, “Stent with Remote Manipulation,” which is hereby incorporated by reference in its entirety.

### TECHNICAL FIELD

The present disclosure relates generally to the field of implantable medical appliances, including stents. More particularly, some embodiments relate to the remote manipulation of a stent, such as a vascular stent, in a compressed configuration or an expanded configuration.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 illustrates a perspective view of a vascular stent in a compressed configuration according to one embodiment of the present disclosure.

FIG. 2 illustrates a perspective view of the vascular stent of FIG. 1 in an expanded configuration.

FIG. 3A illustrates a longitudinal cross-sectional view of a vascular stent with a plurality of strings for manipulating the vascular stent according to one embodiment of the present disclosure.

FIG. 3B illustrates a longitudinal cross-sectional view of the vascular stent of FIG. 3A with one of the plurality of strings pulled to open a fenestration with flap according to one embodiment of the present disclosure.

FIG. 3C illustrates a longitudinal cross-sectional view of the vascular stent of FIG. 3A with one of the plurality of strings pulled to open a fenestration without a flap according to one embodiment of the present disclosure.

FIG. 3D illustrates a longitudinal cross-sectional view of the vascular stent of FIG. 3B with one of the plurality of strings removed without opening a fenestration.

FIG. 4A illustrates a perspective view of a vascular stent with a frame string according to one embodiment of the present disclosure.

FIG. 4B illustrates a perspective view of the vascular stent of FIG. 4A with the frame string pulled to manipulate the shape of a frame of the vascular stent.

FIG. 5A illustrates a side view of a vascular stent with a plurality of shifting strings deployed in a body lumen of a patient according to one embodiment of the present disclosure.

FIG. 5B illustrates a side view of the vascular stent of FIG. 5A with one of the plurality of shifting strings pulled to migrate the vascular stent in a first direction.

FIG. 6 illustrates a perspective view of a bifurcated vascular stent according to one embodiment of the present disclosure.

FIG. 7A illustrates an end view of one embodiment of the primary stent graft of FIG. 7 with a pocket having a circular cross-sectional shape.

FIG. 7B illustrates an end view of another embodiment of the primary stent graft of FIG. 7 with a pocket having a D-shape cross-section.

FIG. 8A is a longitudinal cross-sectional view of the primary stent graft of FIG. 7 with a string.

FIG. 8B is a longitudinal cross-sectional view of the primary stent graft of FIG. 9A with the string pulled to open a fenestration.

FIG. 9A is a longitudinal cross-sectional view of the primary stent graft of FIG. 6 disposed in a diseased aorta.

FIG. 9B is a longitudinal cross-sectional view of the bifurcated vascular stent of FIG. 6 disposed in the diseased aorta.

### DETAILED DESCRIPTION

The components of the embodiments as generally described and illustrated in the figures herein can be arranged and designed in a wide variety of different configurations. Thus, the following more detailed description of various embodiments, as represented in the figures, is not intended to limit the scope of the present disclosure, but is merely representative of various embodiments. While various aspects of the embodiments are presented in drawings, the drawings are not necessarily drawn to scale unless specifically indicated.

The phrase “coupled to” is broad enough to refer to any suitable coupling or other form of interaction between two or more entities, including mechanical and fluidic interaction. Thus, two components may be coupled to each other even though they are not in direct contact with each other. The phrases “attached to” or “attached directly to” refer to interaction between two or more entities which are in direct contact with each other and/or are separated from each other only by a fastener of any suitable variety (e.g., mounting hardware or an adhesive). The phrase “fluid communication” is used in its ordinary sense, and is broad enough to refer to arrangements in which a fluid (e.g., a gas or a liquid) can flow from one element to another element when the elements are in fluid communication with each other.

The terms “proximal” and “distal” are opposite directional terms. For example, the distal end of a device or component is the end of the component that is furthest from the practitioner during ordinary use. The proximal end refers to the opposite end, or the end nearest the practitioner during ordinary use.

The present disclosure includes systems, components, and elements configured to facilitate remote manipulate of a medical appliance. Specific examples given herein reciting elements of a vascular stent are also applicable to other types of stent and other medical appliances. For example, disclosure recited in connection with a vascular stent may be analogously applied to other stents and stent grafts, including tracheal or other airway stents, gastro-intestinal stents including esophageal, bile, and so forth, as well as other medical appliances.

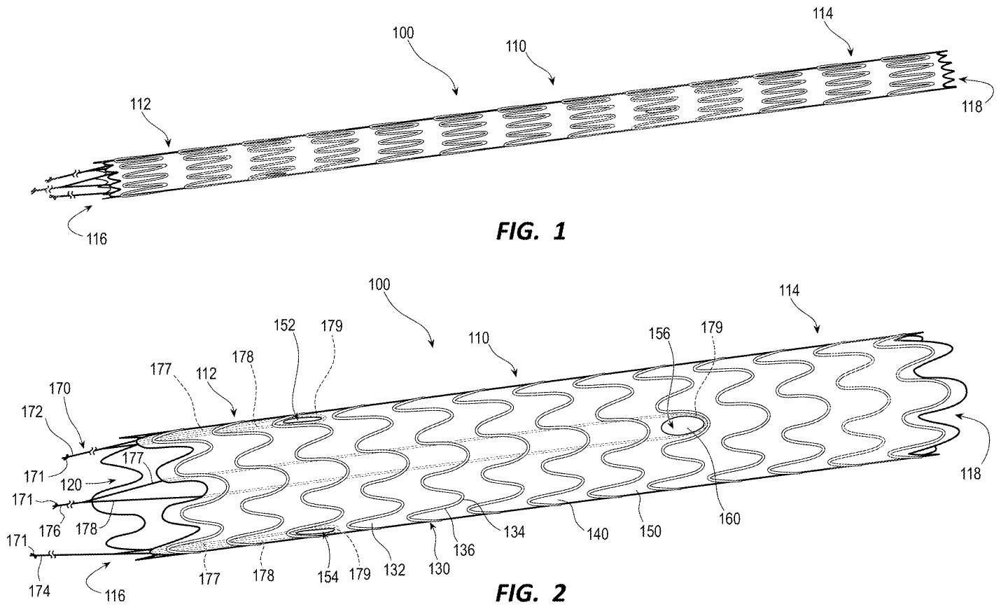

FIG. 1 illustrates a perspective view of a vascular stent 100 in a compressed configuration according to one embodiment of the present disclosure. The vascular stent 100 has a body 110 with a tubular structure with a first end portion 112, a second end portion 114, a first opening or first open end 116, a second opening or second open end 118, and a lumen 120 that extends from the first opening 116 to the second opening 118. In the illustrated configuration, the vascular stent 100 is compressed or crimped into a compressed configuration such that the vascular stent 100 may be loaded into a delivery catheter (not shown). The compressed configuration of the vascular stent 100 may have a relatively low-profile so that the vascular stent 100 fits within a lumen or pod of the delivery catheter. In the compressed configuration, the vascular stent 100 may be delivered to a predetermined location in the patient's vasculature to be deployed or expanded in the predetermined location in the patient's vasculature.

The present disclosure is directed to the remote manipulation of the vascular stent 100 when it is in either a compressed configuration preloaded in the delivery catheter or when the vascular stent 100 is in a deployed or expanded configuration in the vasculature of the patient.

FIG. 2 illustrates the vascular stent 100 in a deployed or expanded configuration. The body 110 of the vascular stent 100 may include a scaffolding structure or frame 130 and a covering 140 disposed over at least a portion of the frame 130. The frame 130 is configured to resist radial compression when the vascular stent 100 is disposed in a body lumen of a patient. In some embodiments, the frame 130 may consist of a single continuous wire 132 forming a plurality of helixes that wrap around forming the lumen 120 of the body 110. In some embodiments, the frame 130 may comprise more than one wire 132. The wire 132 may be comprised of Nitinol (ASTM F2063), or other suitable materials.

In the illustrated embodiment of FIG. 2, the wire 132 may be shaped in a wave-type configuration, the waves defining apexes 134 and arms 136 of the frame 130. The length of each arm 136 of the wire 132 may vary in length and may vary in length along a longitudinal length of the frame 130 itself. The apexes 134 are longitudinally separated along the longitudinal length of the body 110. Along some portions of the body 110, the adjacent helixes of the wire 132 in the longitudinally direction are evenly spaced. Along other portions of the body 110, the space between adjacent helixes in the longitudinal direction are not evenly spaced. The wire 132 may have a variety of different shapes and sizes to form the frame 130 to support the body 110 of the vascular stent 100.

Additionally, the frame 130 may be configured to avow the entire vascular stent 100 to be crimped into a relatively low-profile configuration for delivery, as illustrated in FIG. 1. For example, devices of a certain diameter or constrained profile are more feasible for delivery at certain vascular or other access points than others. For example, in many instances, a device configured for insertion via the radial artery may be relatively smaller than devices configured for insertion via the generally larger femoral artery. A frame may be configured to be crimped into a particular profile to enable potential access at various or desired access points. Similarly, devices having no frame may be configured to be disposed in a particular profile to facilitate access and delivery. Once a device is positioned within the body it may be expanded or deployed in a number of ways, including use of self-expanding materials and configurations. Additionally, some configurations may be designed for expansion by a secondary device, such as a balloon.

As discussed above, the body 110 comprises a covering 140 that at least partially covers the frame 130. In the illustrated embodiment of FIG. 2, an outer portion of the frame 130 is visible and an inner portion of the frame 130 may be encompassed in the covering 140. In some embodiments, the frame 130 may be completely disposed within the covering 140. In some embodiments, the inner portion of the frame 130 may be disposed out of the covering 140 and an outer portion of the frame 130 is disposed within the covering 140. In some embodiments, the covering 140 may comprise multiple layers, including embodiments wherein the frame 130 is disposed or “sandwiched” between layers. The covering 140 may be a polymer, multiple layers of the same polymer, or layers of distinct polymers used in combination.

The covering 140 may include an outer layer or first layer 150 and an inner layer or second layer 160, The outer layer 150 and the inner layer 160 may be coupled together in a variety of different manners, such as an adhesive, welding, and the like. In the illustrated embodiment of FIG. 2, the outer layer 150 extends along the entire length of the vascular stent 100 and the frame 130. The outer layer 150 may define a plurality of apertures 152, 154, 156. For ease of illustration and description, the outer layer 150 defines three distinct apertures, a first aperture 152, a second aperture 154, and a third aperture 156. However, the present disclosure is not so limited and the outer layer 150 may define more than or less than three apertures. The apertures 152, 154, 156 may be placed in a predetermined locations on the vascular stent 100. For example, various apertures may be disposed in a predetermined location that corresponds to a potential side vessel based on the location where the vascular stent 100 is to be implanted.

In the illustrated embodiment, the first aperture 152 and the second aperture 154 are disposed in the same longitudinal location of the vascular stent 100 and are spaced radially apart. The first aperture 152 and the second aperture 154 are disposed on the first end portion 112 of the vascular stent 100. The third aperture 156 is disposed in a different longitudinal location of the vascular stent 100 in the second end portion 114 of the vascular stent 100. The shape and size of each aperture 152, 154, 156 may vary. In the illustrated embodiment, all three apertures 152, 154, 156 each have an oval shape, but the apertures by circular, square, triangular, and the like. The size of the apertures may also be different than illustrated such that they may be bigger or smaller than illustrated. In some embodiments, the aperture 152, 154, 156 may be shaped differently and sized differently from each other or they may be the same size and shape.

Each of the apertures 152, 155, 156 in the illustrated embodiment are sealed closed to the lumen 120 by the inner layer 160. The inner layer 160 may comprises a plurality of distinct pieces or patches that are configured to seal one or more of the plurality of apertures 152. The inner layer 160 may be operable at a corresponding location between the one of the plurality of apertures 152 and the inner layer 160 to create a corresponding fenestration that extends through the covering 140 to the lumen 120. This process may be repeated as many times as there are apertures to open a plurality of fenestrations. However, not all of the apertures need to be opened, only the apertures that the medical practitioner chooses. In other words, the apertures may be selectively openable by a practitioner, depending on the therapeutic needs. One embodiment of opening the fenestrations is described in more detail in relation to RCS. 3A-3D.

The vascular stent 100 may further comprises a plurality of sutures or strings 170. The plurality of strings 170 may be used to open corresponding fenestrations. As illustrated in FIG. 2, a first string 172 corresponds with the first aperture 152, a second string 174 corresponds with the second aperture 154, and a third string 176 corresponds to the third aperture 156. Each string comprises a first end 177, a second end 178, and a looped portion 179 disposed between the first end 177 and the second end 178. The first end 177 and the second end 178 may be coupled together. In the illustrated embodiment, the first end 177 and the second end 178 of each string 172, 174, 176 are tied together at knot 171. However, the first end 177 and the second end 178 may be coupled together in a variety of different manners.

The looped portion 179 of each string 172, 174, 176 is partially looped around the corresponding aperture 152, 154, 156. A portion of the looped portion 179 of the each string 172, 174, 176 may be partially disposed between the outer layer 150 and the inner layer 160 but each string 172, 174, 176 may freely slide between the outer layer 150 and the inner layer 160. As discuss above, the outer layer 150 and the inner layer 160 may be coupled together, via an adhesive or like, except for where a corresponding string slides between the outer layer 150 and the inner layer 160. The outer layer 150 is coupled to the inner layer 160 within the corresponding string such that the apertures are sealed and are coupled on the outside of the corresponding string as well.

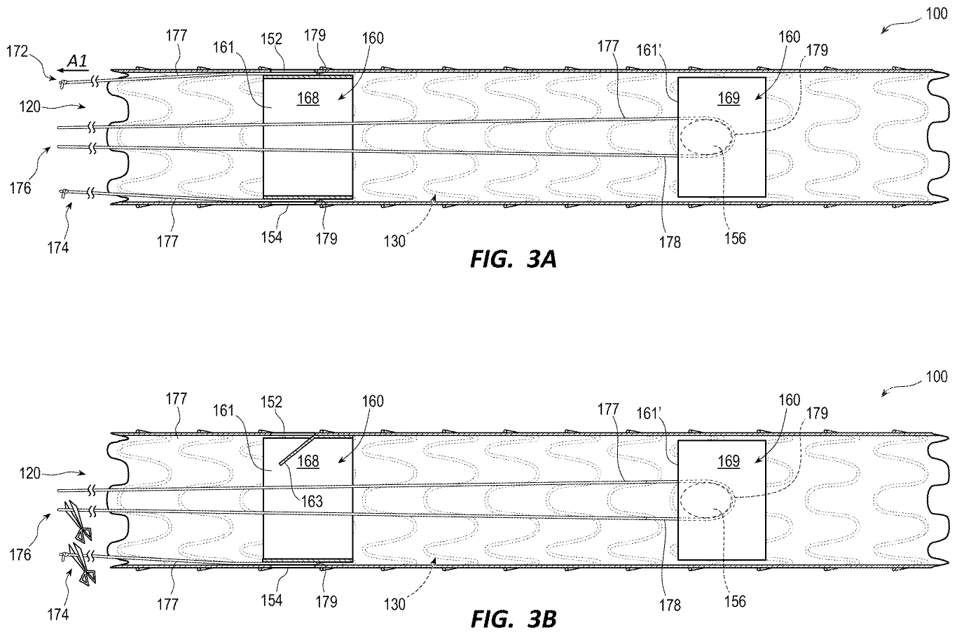

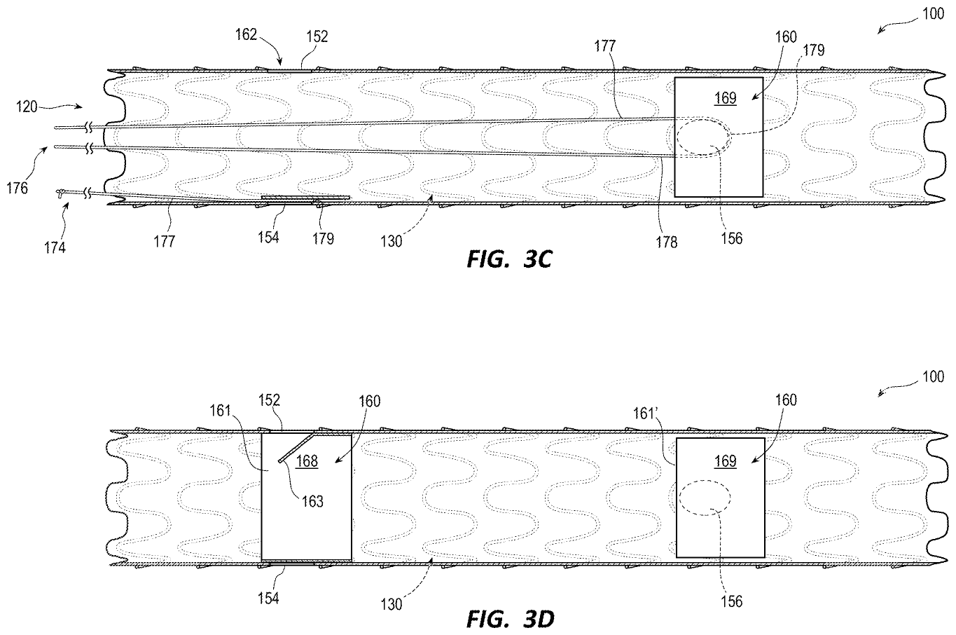

FIG. 3A-3D illustrate a process of manipulating a vascular stent 100 by opening a fenestration through the covering 140. While FIGS. 3A-3D shows the manipulation of the vascular stent 100 in an expanded configuration, such as when the vascular stent 100 is deployed in a vessel, the vascular stent 100 may be manipulated when the vascular stent 100 is in a crimped or compressed configuration. In some embodiments, the vascular stent 100 may be manipulated in a compressed configuration pre-loaded in a delivery catheter. FIG. 3A illustrates a longitudinal cross-sectional view of the vascular stent 100. As discussed above, the vascular stent 100 includes the covering 140 with the outer layer 150 that defines the apertures 152, 154, 156 and the inner layer 160. In the illustrated embodiment, the inner layer 160 comprises a plurality of distinct pieces or patches. For example, in the illustrated embodiment, the inner layer 160 includes a first piece 168 and a second piece 169. The first piece 168 covers and seals the first aperture 152 and the second aperture 154 while the second piece 169 seals the third aperture 156. A longitudinal edge 161 of the first piece 168 is disposed close to the circumference of the first aperture 152 and the circumference of the second aperture and the longitudinal edge 161′ of the second piece 169 is disposed close to the circumference of the third aperture 156. None of the fenestrations have been opened in FIG. 3A as all of the apertures are sealed by the inner layer 160.

The vascular stent 100 further includes the plurality of strings 170, with the first string 172 partially looped around the first aperture 152, the second string 174 partially looped around the second aperture 154, and the third string 176 partially looped around the third aperture 156. FIG. 3A clearly shows that the looped portion 179 of the third string 176 partially looped around the third aperture 156.

The plurality of strings 170 may be used to open the fenestrations. For example, in FIG. 3A, a user may grasp the first end 177 and the second end 178 of the first string 172 and pull both ends 177, 178 in the same direction, the direction of the arrow A1. As the user pull both ends 177, 178 of the first string 172, the first string 172 decouples or sever the coupling between the outer layer 150 and the inner layer 160 thereby opening a fenestration 162 that extends through the cover 140 to the lumen 120 through the first aperture 152. An edge 161 of the inner layer 160 is disposed close to the edge to the aperture 152 so that the first string 172 does not have to travel far to open the fenestration 162 between the first aperture 152 and the lumen 120.

In some embodiments, the opening of the fenestration 162 may create a flap 163 in the inner layer 160 as illustrated in FIG. 3B. In some embodiments, the opening of the fenestration 162 tears away the inner layer 160 that an opening is created in the inner layer 160 that corresponds to the first aperture 152 such that there is no flap and the fenestration 162 is opened directly to the lumen 120 as illustrated in FIG. 3C. The inner layer 160 may have weakened portions, frangible portion, or perforations to assist in a clean or complete breakaway of the inner layer 160 from the outer layer 150 at the corresponding aperture to form the fenestration. Additional fenestrations may be opened in a similar manner at corresponding apertures 154, 156.

After the user has opened all of the desired fenestrations in the vascular stent 100, the remaining plurality of strings 170 may be removed from the vascular stent 100. For example, the user may decouple the first end 177 from the second end 178 on the second string 174 and then pull on one of the first end 177 or the second end 178 of the second string 174 to slide the second string 174 between the outer layer 150 and the inner layer 160 to remove the second string 174 form the vascular stent 100. The third string 176 may also be removed in a similar manner.

The decoupling of the first end 177 to the second end 178 by cutting with scissors either the first end 177 or the second end 178. FIG. 3B illustrates the second string 174 and the third string 176 being cut with scissors. FIG. 3D illustrates the vascular stent 100 with fenestration 162 opened and the remaining strings 174, 176 removed without creating a corresponding fenestration between the outer layer 150 and the inner layer 160.

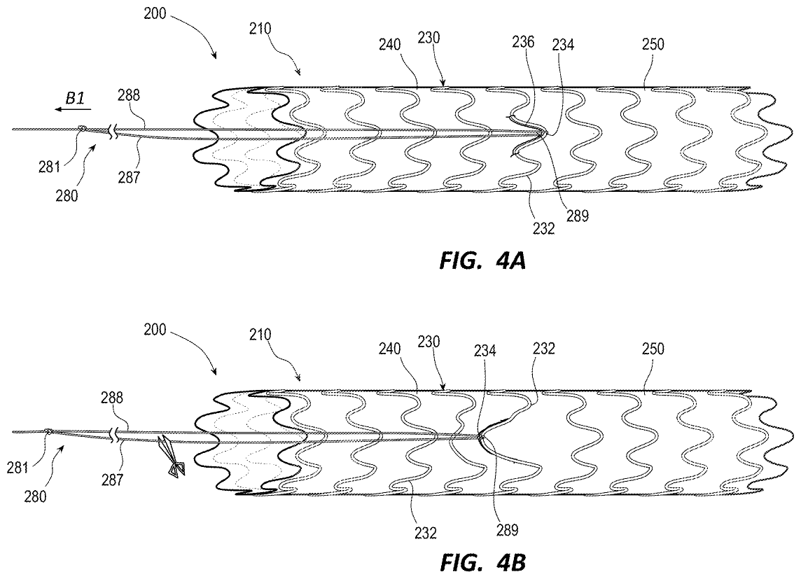

FIGS. 4A and 4B depicts an embodiment of a vascular stent 200 that resembles the vascular stent 100 described above in certain respects. Accordingly, like features are designated with like reference numerals, with the leading digits incremented to “2.” For example, the embodiment depicted in FIGS. 1-3D includes a body 210 that may, in some respects, resemble the body 110 of FIGS. 1-3D. Relevant disclosure set forth above regarding similarly identified features thus may not be repeated hereafter. Moreover, specific features of the vascular stent 100 and related components shown in FIGS. 1-3D may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the vascular stent 200 and related components depicted in FIGS. 4A and 4B. Any suitable combination of the features, and variations of the same, described with respect to the vascular stent 100 and related components illustrated in FIGS. 1-3D can be employed with the vascular stent 200 and related components of FIGS. 4A and 4B, and vice versa. This pattern of disclosure applies equally to further embodiments depicted in subsequent figures and described hereafter, wherein the leading digits may be further incremented.

FIGS. 4A and 4B illustrate a process of manipulating the vascular stent 200 by changing the shape of a frame 230. While FIGS. 4A and 4B shows the manipulation of the vascular stent 200 in an expanded configuration, such as when the vascular stent 200 is deployed in a vessel, the vascular stent 200 may be manipulated when the vascular stent 200 is in a crimped or compressed configuration. In some embodiments, the vascular stent 200 may be manipulated in a compressed configuration pre-loaded in a delivery catheter.

FIG. 4A illustrates a side view of the vascular stent 200 with a frame string 280. For ease of illustration and disclosure, only a single frame string 280 is illustrated. However, the present disclosure is not so limited and there may be more than one frame string 280 coupled to the frame 230 of the vascular stent 200. The frame string 280 comprises a first end 287, and second end 288, and a looped portion 289 disposed between the first end 287 and the second end 288. The first end 287 and the second end 288 may be coupled together. In the illustrated embodiment, the first end 287 and the second end 288 of the frame string 280 are tied together at knot 281. However, the first end 287 and the second end 288 may be coupled together in a variety of different manners.

The looped portion 289 of the frame string 280 may be wrapped around a portion of the frame 230. For example, the frame 230 may be fabricated of a single wire 232 that may be shaped in a wave-type configuration, the waves defining apexes 234 and arms 236 of the frame 230. In the illustrated embodiment, the looped portion 289 of the frame string 280 is coupled to an apex 234 of the frame 230.

The frame string 280 may be used to manipulate the shape of the frame 230. For example, in FIG. 4A, a user may grasp the first end 287 and the second end 288 of the frame string 282 and pull both ends 287, 288 in the same direction, the direction of the arrow B1, for example. As the user pull both ends 287, 288 of the frame string 280, the frame string 280 pulls on the apex 234 to shift the direction of the apex 234 from a first direction to a second direction opposite the first direction so that the apex 234 is moved closer to an adjacent apex 234, thus changing the shape of the frame 230 as illustrated in FIG. 4B.

This may be useful in situations where a bigger fenestration may be needed and the user want to make a larger area by manipulating the frame 230 to make a larger area. As discussed above, an aperture in an outer layer 250 of the covering 240 may already located in the area but it needs the frame 230 to be manipulated so that the frame 230 is not in the way to make the bigger fenestration with the aperture in the outer layer 250. A string similar to the strings discussed in relation to FIGS. 3A-3D may be used to open a fenestration.

While the illustrated embodiment, only illustrated the frame 230 manipulated in a single way, the frame 230 may be manipulated in a number of different ways. For example, entire helixes may be moved so that they are closer to an adjacent helix and so forth.

After the user has finished manipulating the frame 230 of the vascular stent 200, the frame string 280 may be removed from the vascular stent 200. For example, the user may decouple the first end 287 from the second end 288 on the frame string 280 and then pull on one of the first end 287 or the second end 288 of the frame string 280 to slide the frame string 280 off of the frame 230 of the vascular stent 200. The decoupling of the first end 287 to the second end 288 may be done by cutting with scissors either the first end 287 or the second end 288 as shown in FIG. 4B.

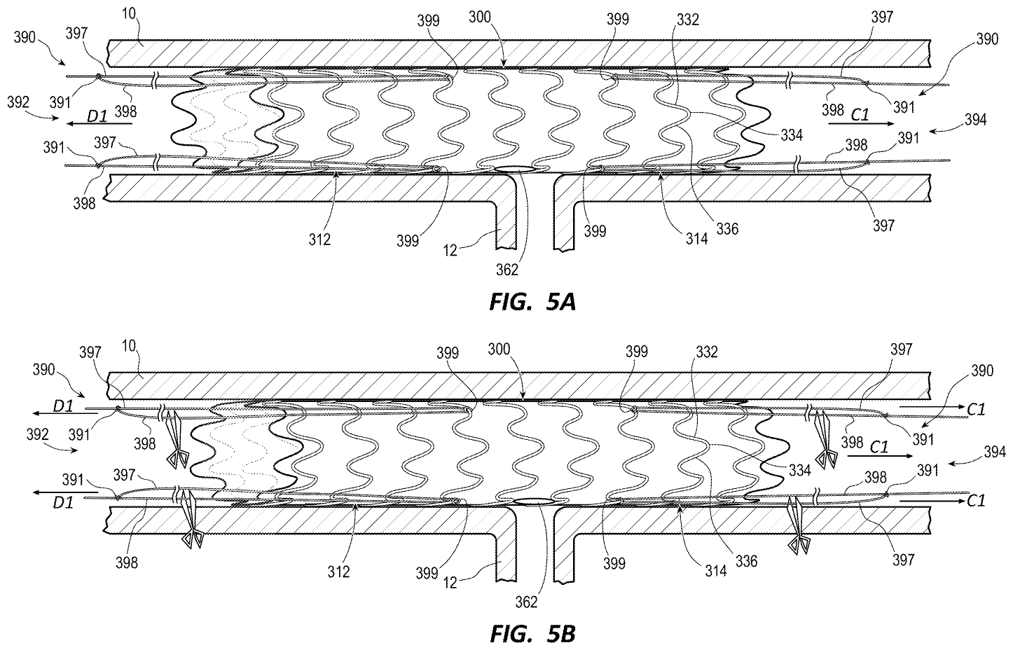

FIGS. 5A and 5B illustrate a process of moving or shifting a vascular stent 300 in a first direction or a second direction when the vascular stent 300 is deployed in a vessel 10. During placement of the vascular stent 300, the fenestrations in the vascular stent 300 may not aligned with side vessels as envisioned by the user. Accordingly, the user may shift the vascular stent 300 relative to the vessel 10 to align the fenestrations of the vascular stent 300 with any side vessels at the location of deployment.

FIG. 5A illustrates a side view of the vascular stent 300 with a plurality of shifting strings 390. For ease of illustration and disclosure, four shifting strings 390 are illustrated. However, the present disclosure is not so limited and there may be more than or less than four shifting strings 390 coupled to the frame 330 of the vascular stent 300.

The shifting strings 390 may be grouped into directional shifting strings groups that are configured to shift the vascular stent 300 in different directions. For example, a first group of shifting strings 392 are coupled to a first end portion 312 and a second group of shifting strings 394 are coupled to a second end portion 314. Each shifting string 390 comprises a first end 397, and second end 398, and a looped portion 399 disposed between the first end 397 and the second end 398. The first end 397 and the second end 398 may be coupled together. In the illustrated embodiment, the first end 397 and the second end 398 of each shifting string 390 are tied together at knot 391. However, the first end 397 and the second end 398 may be coupled together in a variety of different manners.

The looped portion 399 of each shifting string 390 may be wrapped around a portion of the frame 330. For example, the frame 330 may be fabricated of a single wire 332 that may be shaped in a wave-type configuration, the waves defining apexes 334 and arms 336 of the frame 330. In the illustrated embodiment, the looped portion 389 of the each shifting string 390 is coupled to an apex 334 of the frame 330.

The plurality of shifting strings 390 may be used to shift the location of the vascular stent 300 in the vessel 10. For example, in FIG. 5A, vascular stent 300 is deployed, but the fenestration 262 is not aligned with the side vessel 12. A user may grasp the first end 397 and the second end 398 of the second group of shifting string 394 to shift the vascular stent 300 in the direction of the arrow C1. As the user pull both ends 397, 398 of the second group of shifting string 394, the second group of shifting strings 394 pulls on the vascular stent 300 to shift the vascular stent 300 in the direction of the arrow C1. The user may shift the vascular stent 300 until the fenestration 362 aligns with the side vessel 12 as illustrated in FIG. 5B.

If the user over shifts the vascular stent 300 and the fenestration 263 goes past the side vessel 12, the user may use the first group of shifting string 392 to shift the vascular stent 100 in the direction of the arrow D1 to align the fenestration 362 with the side vessel 12.

After the user has finished shifting the vascular stent 300 relative to the vessel 10, the plurality of shifting strings 390 may be removed from the vascular stent 300. For example, the user may decouple the first end 397 from the second end 398 on each shifting string 390 and then pull on one of the first end 397 or the second end 398 of each shifting string 390 to slide each shifting string 390 off of the frame 330 of the vascular stent 300. The decoupling of the first end 397 to the second end 398 may be done by cutting with scissors either the first end 397 or the second end 398.

In some embodiments that use the shifting strings 390, the procedure may use two different access points. The first group of shifting strings 392 may use a first access point and the second group of shifting strings 394 may use a second access point different from the first access point. Accordingly, the user may access the plurality of shifting strings 390 from the different access points to achieve pulling the vascular stent 300 in different directions.

In this process, a user may advance the vascular stent 300 through the first access point to the predetermined location in the patient vascular. Before the vascular stent is advanced, a guide wire coupled to the second group of shifting stent 294 may be advanced through from the first access point to the predetermined deployed location and then advanced to the second access point so that the second group of shifting strings 394 extend out of the second access point. The vascular stent 300 may be advanced simultaneously as the second group of shifting strings 394 or after the second group of shifting strings 394 extend out of the second access point. The second group of shifting strings 394 may have sufficient length to extend from at least the first access point to the second access point.

In some embodiments, the vascular stent may come pre-loaded in a delivery catheter (not shown) in a kit. The vascular stent may come with a variety of strings for manipulating the vascular stent when the vascular stent is pre-loaded in the delivery catheter or after the vascular stent is delivered to the vasculature of the patient. For example, the vascular stent may include the plurality of strings used to open fenestrations in the vascular stent. The vascular stent may also include a plurality of frame strings to manipulate the frame of the vascular stent. The vascular stent may also include a plurality of shifting strings to shift the vascular stent in the vasculature after the vascular stent has been deployed.

The kit may also include a map or key for the user to use to manipulate the vascular stent. Each string attached to the vascular stent may be label with indicia (such as a number) and the map indicates to the user the purpose of each string. For example, string 1 may be for opening a fenestration in a specific location on the vascular stent. String 2 may be for opening another fenestration in another specific location on the vascular stent. String 3 may be for manipulating the frame and changing the shape of the frame of the vascular stent. The user may manipulate the vascular stent to fit the specific uses of the specific patient the vascular stent will be used. String 4 may be for shifting the vascular stent in a specific direction after deployment and String 5 may be for shifting the vascular stent in an opposite direction. The map may be a piece of paper of with an enlarged view of the stent and all the strings and provide a chart that outlines the purpose of each string.

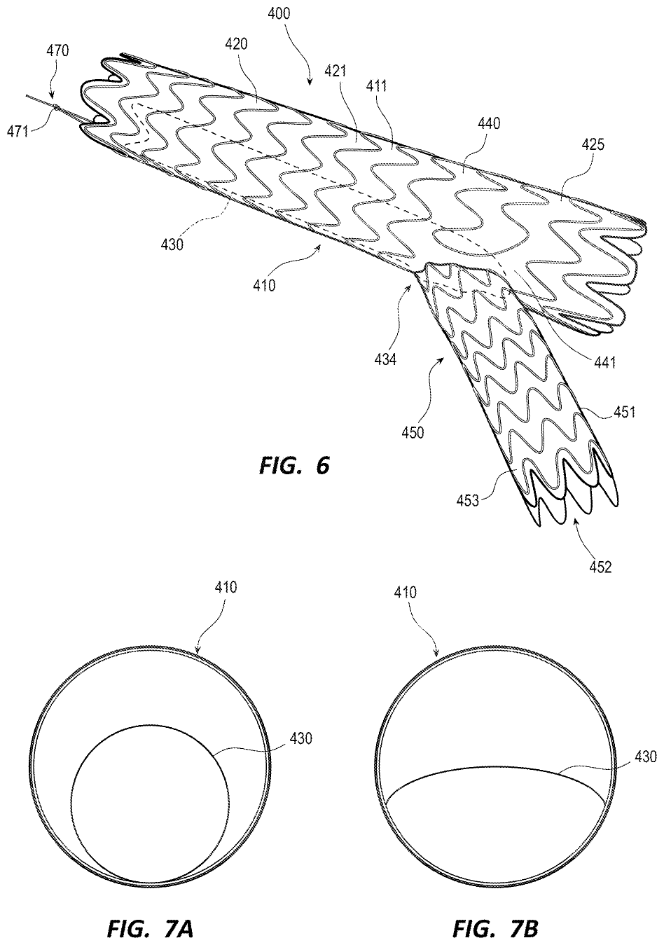

FIG. 6A illustrates an embodiment of a bifurcated vascular prosthesis 400. In the illustrated embodiment, the bifurcated vascular prosthesis 400 is partially composed of a primary stent graft 410 and a secondary stent graft 450 selectively couplable to the primary stent graft 410. As illustrated, the embodiment of the bifurcated vascular prosthesis 400 may be sized or otherwise configured to repair a diseased aorta vessel proximal to a bifurcation of iliac arteries. In various other embodiments, the bifurcated vascular prosthesis 400 can be configured to repair any diseased arterial or venous vessel, including those including a bifurcation, such as a coronary artery, a carotid artery, a popliteal artery, a common femoral artery, brachiocephalic vein, etc. The bifurcated vascular prosthesis 400 may be placed in the arterial vascular system such that blood flow through the bifurcated vascular prosthesis 400 splits into two or more vessels. For example, the bifurcated vascular prosthesis 400 may be deployed at a bifurcation between an aorta vessel and the left and right iliac vessels. The bifurcated vascular prosthesis 400 may also be placed in the venous vascular system such that blood flow through the bifurcated vascular prosthesis 400 converges into a single vessel from two or more vessels. For example, the bifurcated vascular prosthesis 400 may be deployed at the bifurcation between a superior vena cava and left and right brachiocephalic vessels. As noted above, disclosure here regarding treatment of a specific region, such as the aorta, can be analogously applied to treatment of other portions of the vasculature or other lumens of the body.

The primary stent graft 410 includes a body 411 having a proximal portion 420 and a distal portion 425. The body 411 may be generally cylindrical in shape having a bore 412 defined by a wall 423 extending through the proximal and distal portions 420, 425, such that blood can flow from the aorta, through the bore 412, and into an iliac artery when the bifurcated vascular prosthesis 400 is implanted. The body 411 may be formed of a variety of materials and/or layers of materials, including biocompatible materials that are resistant to passage of blood through the wall 423. For example, the biocompatible material may be polyethylene terephthalate, polyurethane, silicone rubber, nylon, or fluoropolymer. Other biocompatible materials are contemplated within the scope of this disclosure. A thickness of the wall 423 may range from about 0.07 millimeter to about 0.5 millimeter.

In some embodiments, a length of the body 411 may range from about 50 mm to about 250 mm with a length of the proximal portion 420 ranging from about 20% to about 80% of the length of the primary stent graft 410. An outer diameter of the body 411 may range from about 18 millimeters to about 55 millimeters. In one embodiment, the body 411 may include a flared proximal end to facilitate sealing of the proximal portion 420 with a wall of the aorta and to prevent leakage of blood between the proximal portion 420 and the aorta wall. In some embodiments, the body 411 may include a cuff disposed adjacent the proximal portion 420 configured to facilitate sealing of the proximal portion 420 with the vessel wall and to prevent leakage of blood between the proximal portion 420 and the aorta wall. In other embodiments, the body 411 may include fixation features configured to prevent migration of the bifurcated vascular prosthesis 400 relative to the aorta wall. The fixation features may include protruding barbs, sharpened protruding barbs, an adhesive, inflatable portions, strut hooks, etc.

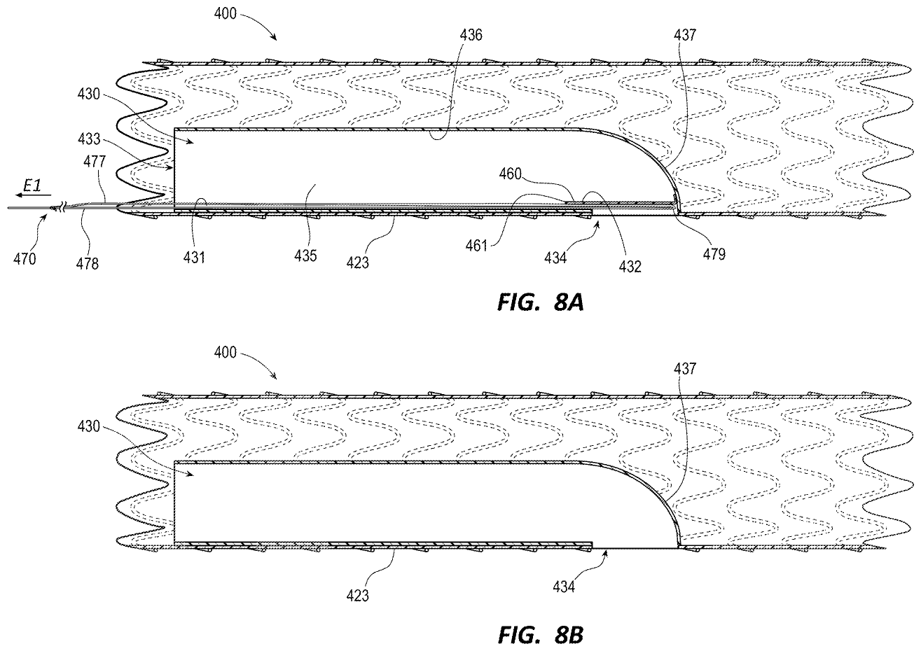

The proximal portion 420 includes a pocket 430 disposed within the bore 412 and configured to receive the secondary stent graft 450, as illustrated in FIGS. 8A and 8B. The pocket 430 is oriented such that it extends in a proximal direction along the wall 423 of the bore 412. A portion of a wall 436 of the pocket 430 may be coupled to the wall 423. The pocket 430 may be integrally formed with the wall 423 of the body 411. The proximal portion 420 and the pocket 430 may be formed to be an integral or unibody component such that there is not a seam or joint at a junction of the body 411 and the pocket 430. The pocket 430 includes a proximal end 431, a distal end 432, a proximal opening 433, a distal opening 434, and a lumen 435 defined by a wall 436. The distal opening 434 may be sealed before the secondary stent graft is inserted into the distal opening 434. The distal opening 434 may be opened using a string 470, as discussed in relation to FIGS. 8A and 8B. In some embodiments, the distal opening 434 may be disposed at any location along a length of the proximal portion 420. A diameter of the distal opening 434 may be sized to receive the secondary stent graft 450. In other words, the distal opening 434 may be correlated to the secondary stent graft 450, for example, the diameter of the distal opening 434 may be equivalent to or smaller than an outer diameter of the secondary stent graft 450.

The pocket 430 may have a substantially round transverse cross-sectional shape, as shown in FIG. 7A. In another embodiment, a transverse cross-section of the pocket 430 may include a D-shape, as shown in FIG. 7B. In other embodiments, the pocket 430 may include any suitable transverse cross-sectional shape, such as oval, obround, semicircular, D-shaped, etc.

In some embodiments, the pocket 430 may be formed of the same material as the body 411 while in other embodiments these elements may be formed of different materials. A length of the pocket 430 may range from about 5 mm to about 50 mm. A thickness of the wall 436 may range from about 0.1 millimeter to about 0.9 millimeter and from about 0.21 millimeter to about 0.57 millimeter. The proximal end 431 of the pocket 430 is disposed distally of a proximal end of the body 411. The proximal opening 433 is disposed at the proximal end 431. The distal opening 434 is disposed adjacent the distal end 432 and in the wall 123 of the body 411. The lumen 435 extends from the proximal opening 433 to the distal opening 434. The lumen 435 may be configured to sealingly receive the secondary stent graft 450. A diameter of the lumen 435 may be equivalent to or smaller than an outer diameter of the secondary stent graft 450 such that an outer surface of the secondary stent graft 450 seals with an inner surface of the wall 436 of the pocket 430. In certain embodiments, the wall 436 may be circumferentially stretched when the secondary stent graft 450 is disposed within the lumen 435.

As shown in FIG. 6, a wire scaffolding, framework, or stent such as wire stent 440 is shown to circumferentially surround the body 411. The wire stent 440 may be configured to radially expand the body 411 from a crimped or delivery configuration to an expanded or deployed configuration. When the bifurcated vascular prosthesis 400 is deployed within a blood vessel, the body 411 may be pressed against a wall of the blood vessel. The wire stent 440 may be formed of any suitable material such as nickel-titanium alloy, stainless steel, platinum, polymers, etc. The wire stent 440 may have a zig-zag pattern, a wave pattern, or any other suitable pattern. An area 441 of the body 411 surrounding the distal opening 434 may be void of the wire stent 440. In the void area 441, the zig-zag pattern may loop back on itself to prevent the wire stent 440 from crossing over the distal opening 434. The wire stent 440 may be pre-formed or formed over the body 411. The material, pattern, and wire diameter of the wire stent 440 may be configured to provide a chronic radially outward directed force and a resistance to a radially inward directed force.

The secondary stent graft 450 includes a body 451 including a proximal portion 456 and a distal portion 457. The body 451 may be generally cylindrical in shape having a bore 452 defined by a wall 453 such that blood can flow from the aorta, through the bore 452, and into an iliac artery when the bifurcated vascular prosthesis 400 is implanted. A cross-sectional area of the bore 452 may be substantially equivalent to a cross-sectional area of the bore 412 of the primary stent graft 410. This configuration facilitates substantially equivalent blood flow rates through the bores 412, 452 such that blood flow to the iliac arteries is substantially equivalent.

The body 451 may be formed of a variety of materials and/or layers of materials, including biocompatible materials that are resistant to passage of blood through the wall 453. For example, the biocompatible material may be polyethylene terephthalate, polyurethane, silicone rubber, nylon, or fluoropolymer. Other biocompatible materials are contemplated within the scope of this disclosure. A thickness of the wall 453 may range from about 0.1 millimeter to about 0.9 millimeter and from about 0.21 millimeter to about 0.57 millimeter.

In some embodiments, a length of the body 451 may range from about 20 millimeters to about 250 millimeters. An outer diameter of the body 451 may range from about 3 millimeters to about 55 millimeters.

In certain embodiments, the lumen 435 of the pocket 430 can be inwardly tapered from the proximal end 431 to the distal end 432 and the secondary stent graft 450 can be inwardly tapered along the proximal portion 456 to prevent distal migration of the secondary stent graft 450 relative to the primary stent graft 410. In another embodiment, the body 451 may include a step transition from a larger diameter proximal portion 456 to a smaller diameter distal portion 457. The pocket 430 may include a corresponding step transition to receive the step transition of the body 451 to prevent distal migration of the secondary stent graft 450 relative to the primary stent graft 410.

A wire scaffolding, framework, or stent such as wire stent 455 is shown to circumferentially surround the body 451. The wire stent 455 may be configured to radially expand the body 451 from a crimped or delivery configuration to an expanded or deployed configuration. When the bifurcated vascular prosthesis 400 is deployed, the proximal portion of the body 451 may be pressed against the wall 436 of the pocket 430 and a distal portion of the body 451 may be pressed against a wall of the iliac artery. The wire stent 455 may be formed of any suitable material, such as nickel-titanium alloy, stainless steel, platinum, polymers, etc. The wire stent 455 may have a zig-zag pattern, a wave pattern, or any other suitable pattern. The wire stent 455 may be pre-formed or formed over the body 451. The material, pattern, and wire diameter of the wire stent 455 may be configured to provide a chronic radially outward directed force and a resistance to a radially inward directed force. In some embodiments, the wire stent 455 may include one, two, three, or more lumens.

FIGS. 8A and 8B illustrate a process of opening the distal opening 434 so that the secondary stent graft 450 may be inserted through the distal opening 434 to form the bifurcated vascular prosthesis 400. FIG. 8A illustrates a cross-sectional side view of the primary stent graft 410. The distal opening 434 of the pocket 430 may be disposed in the wall 423. The distal end 432 of the pocket 430 is closed. The distal end 432 may include an end wall 437 disposed at an angle ranging from about 30 degrees to about 90 degrees. The end wall 437 may be curved, as shown in FIG. 8A. The distal opening 434 may be sealed with an inner layer 460, similar to the inner layer 160 discussed in regard to the vascular stent 100.

The string 470 may comprises a first end 477, a second end 478 and a looped portion 479 that partially loops around the distal opening 434. A portion of the string 470 may be partially disposed between the body 411 and the inner layer 460, but each string 470 may freely slide between the body 411 and the inner layer 460.

The string 470 may be used to open the distal opening 434. For example, in FIG. 8A, the user may grasp the first end 477 and the second end 478 of the string 470 and pull both ends 477, 478 in the same direction, the direction of the arrow E1. As the user pull both ends 477, 478 of the string 470, the string 470 decouples or severs the coupling between the body 411 and the inner layer 460 thereby opening the distal opening 434 the body 411 to the bore 412. An edge 461 of the inner layer 460 is disposed close to the edge to the distal opening 434 so that the string 470 does not have to travel far to open the fenestration.

The inner layer 460 may have weakened portions, frangible portions, or perforations to assist in a clean breakaway of the inner layer 460 from the body 411 at the distal opening 434 to form the fenestration.

Once the distal opening 434 is opened, the end wall 437 can be configured to allow the secondary stent graft 450 to extend radially outward from the primary stent graft 410 at an angle ranging from about zero degree to about 180 degrees, as shown in FIG. 6.

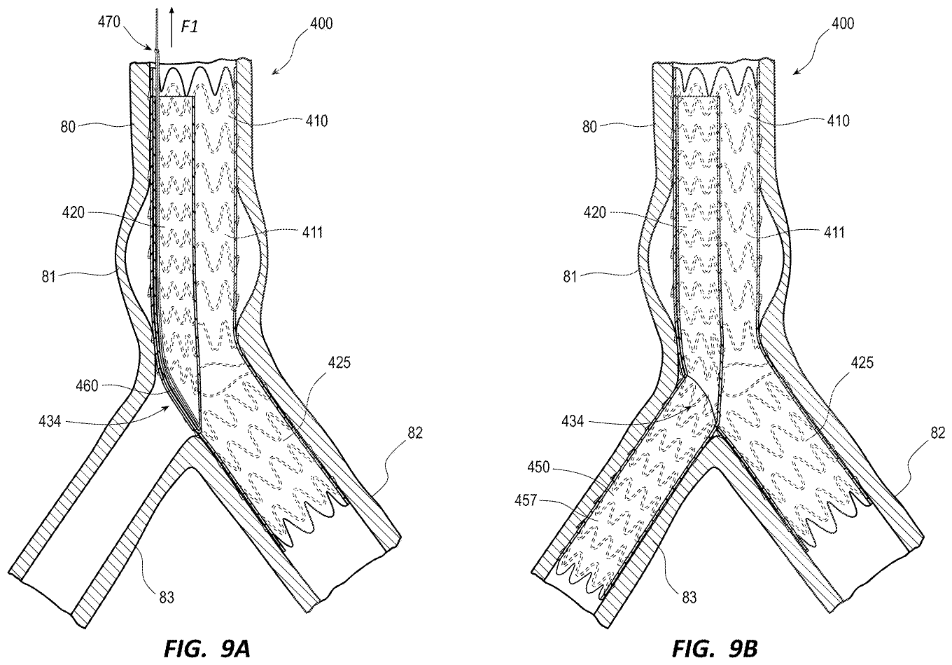

FIGS. 9A-9C illustrate a method of implanting the bifurcated vascular prosthesis 400 in a diseased blood vessel (e.g., aorta) and iliac arteries. FIG. 9A shows the primary stent graft 410 of the bifurcated vascular prosthesis 400 deployed in the aorta 80. The primary stent graft 410 may be deployed using a delivery catheter system, wherein the primary stent graft 410 is radially compressed and disposed within the delivery catheter system. The body 411 may be radially expanded (e.g., self-expanded or balloon expanded) to compress the proximal portion 420 against a healthy tissue section of a wall of the aorta 80 proximal to a diseased section 81 of the aorta 80 such that the bifurcated vascular prosthesis 400 may be secured in place. The diseased section 81 may be an aneurysm, a pseudoaneurysm, an aortic dissection, a stenosis, or any other type of vascular disease. The distal portion 425 may extend distally into a first iliac artery 82 and may be radially expanded to compress against a wall of the first iliac artery 82.

The user may pull on the string 470 in the direction of the arrow F1 to open the distal opening 434 by removing the inner layer 460. FIG. 9C illustrates the distal opening 434 open with the string 470 removed.

FIG. 9C shows the secondary stent graft 450 deployed and coupled to the primary stent graft 410 through the distal opening 434. The secondary stent graft 450 may be deployed using a delivery catheter system, wherein the secondary stent graft 450 is radially compressed and disposed within the delivery catheter system. A proximal portion 456 is disposed within the pocket 430 and a distal portion 457 extends through the distal opening 434 and into the second iliac artery 83. The secondary stent graft 450 may be radially expanded (e.g., self-expanded or balloon expanded) to compress the proximal portion 456 against the wall 436 of the pocket 430 and the distal portion 457 against a wall of the second iliac artery 83 to form a fluid tight seal and to secure the secondary stent graft 450 in place. When the bifurcated vascular prosthesis 400 is fully deployed, as shown in FIG. 9C, blood can flow from the aorta 80, into the primary stent graft 410. Within the primary stent graft 410 the blood flow is divided into two flows, a first flow continues through the primary stent graft 410 and exits into the first iliac artery 82, and the second flow enters the secondary stent graft 450, flows through the secondary stent graft 450, and exits into the second iliac artery 83. The blood flows into the first and second iliac arteries 82, 83 can be substantially equivalent. In other embodiments, the bifurcated vascular prosthesis 400 may include more than two lumens and the blood flow in each of the lumens may be substantially equivalent or may be different depending on a size of blood vessel the lumen is in fluid communication with.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated by one of skill in the art with the benefit of this disclosure that in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. It will be apparent to those having skill in the art that changes may be made to the details of the above-described embodiments without departing from the underlying principles of the present disclosure.

We claim:

1. A vascular stent comprising:
a body comprising a first open end and a second open end with a lumen extending from the first open end to the second open end, the body comprising:
a frame;
a covering disposed on at least a portion of the frame, the covering comprising an outer layer and an inner layer coupled together; and
a plurality of apertures defined in the outer layer of the covering and sealed by the inner layer of the covering,
wherein the inner layer is openable at a corresponding location of each of the plurality of apertures to create a corresponding fenestration that extends through the covering.

2. The vascular stent of claim 1, further comprising a plurality of strings, wherein each string of the plurality of string corresponds to the location of one of the plurality of apertures.

3. The vascular stent of claim 2, wherein a first end and a second end of each string are coupled together and a looped portion of each string is partially looped around one of the apertures of the plurality of apertures and is disposed between the outer layer and the inner layer.

4. The vascular stent of claim 3, wherein the looped portion of each string is configured to freely slide between the outer layer and the inner layer.

5. The vascular stent of claim 3, wherein when the first end and the second end of each string are pulled in a same direction away from the vascular stent, the string is configured to decouple the inner layer from the outer layer to open the corresponding aperture in the outer layer to the lumen of the vascular stent.

6. The vascular stent of claim 2, wherein the inner layer forms a flap at the corresponding aperture in the outer layer when the string is pulled away.

7. The vascular stent of claim 2, wherein the inner layer comprises a frangible portion that aligns with the corresponding aperture of the outer layer such that when the string is pulled away the frangible portion is removed to form the corresponding fenestration.

8. The vascular stent of claim 2, wherein each string of the plurality of strings is removable without creating the corresponding fenestration between the outer layer and the inner layer.

9. The vascular stent of claim 1, further comprising a plurality of frame strings, wherein a first end and a second end of each frame string are tied together and a looped portion of each frame string is coupled to the frame.

10. The vascular stent of claim 9, wherein when the first end and the second end of each frame string are pulled in a same direction away from the vascular stent, the string is configured to manipulate the frame and change a shape of the frame.

11. A method of manipulating a vascular stent preloaded in a catheter comprising:
selecting a first selected string of a plurality of strings that are coupled to a vascular stent preloaded in a catheter, wherein a first end and a second end of each string of the plurality of strings are coupled together and a looped portion of each string is coupled to the vascular stent; and
pulling the first end and the second end of the first selected string in a same direction away from the vascular stent to manipulate the vascular stent.

12. The method of claim 11, wherein pulling the first end and the second end of the first selected string opens a fenestration through a body of the vascular stent in a predetermined location.

13. The method of claim 11, wherein pulling the first end and the second end of the first selected string changes a shape of a frame of the vascular stent.

14. The method of claim 11, further comprising:
decoupling the first end and the second end of all the remaining strings of the plurality of strings after an initial string or strings is pulled; and
pulling on either the first end or the second end of each remaining strings of the plurality of string to decouple and slide the looped portion from the vascular stent.

15. A method of manipulating a vascular stent deployed in a body lumen comprising:
advancing a vascular stent through a first access point to a predetermined location in a vasculature of a patient, wherein the vascular stent comprises a plurality of strings with each string comprising a first end and a second end coupled together and a looped portion coupled to the vascular stent;
deploying the vascular stent in the predetermined location;
selecting a first selected string of the plurality of strings;
pulling the first end and the second end of the first selected string in a same direction away from the vascular stent to manipulate the vascular stent.

16. The method of claim 15, wherein pulling the first end and the second end of the first selected string opens a fenestration through a body of the vascular stent in a predetermined location.

17. The method of claim 15, wherein pulling the first end and the second end of the first selected string changes a shape of a frame of the vascular stent.

18. The method of claim 15, wherein pulling the first end and the second end of the first selected string migrates the vascular stent in the predetermined location within the vasculature in the same direction as the first selected string is pulled.

19. The method of claim 15, wherein at least one of the plurality of strings extends out the first access point and at least one of the plurality of strings extends outside a second access point that is on an opposite side of the vascular stent as the first access point.

20. A vascular stent kit comprising:
a vascular stent comprising:
a body comprising a first open end and a second open end with a lumen extending from the first open end to the second open end, the body comprising:
a frame;
a covering disposed on at least a portion of the frame, the covering comprising an outer layer and an inner layer coupled together;
a plurality of apertures defined in the outer layer of the covering and sealed by the inner layer of the covering; and
a plurality of strings,
wherein a first end and a second end of each string are coupled together and a looped portion of each string is partially looped around one of the apertures of the plurality of apertures and is disposed between the outer layer and the inner layer; and
a map that outlines each string of the plurality of strings and the corresponding purpose of each string.
